(19) United States
(12) Patent Application Publication
(10) Pub. No.: US 2004/0092906 A1
(43) Pub. Date: May 13, 2004

(54) Disposable vaginal cannula for the simultaneous administration of drugs in different forms

(76) Inventor: Luciano Tosato, Rimini (IT)

Correspondence Address: Walter H. Schneider, 21530 Beechwood Rd., Circleville, OH, 43113, US

(21) Appl. No.: 10/679,628

(22) Filed: Sep. 29, 2003

(30) Foreign Application Priority Data
Apr. 5, 2001 (IT) FO2001A000010

(51) Int. Cl. A61M 31/00

(52) U.S. Cl. 604/500

(57) ABSTRACT

A vaginal cannula which allows drugs of different physical forms, such as a gel and a tablet and which must remain separated until the time of use, to be introduced into the vagina simultaneously

This invention relates to a device, in particular a disposable plastic cannula, which enables two drugs presented in different physical forms, in particular a gel and a tablet and/or powder, to be delivered into the vagina simultaneously.

The cannula has a particular configuration which enables it to keep the two products constantly separate at the time of packaging and during storage, until the time of use, to prevent deterioration of one or both substances, such as the vaginal tablet, which must not be rehydrated before use, because rehydration causes loss of its therapeutic efficacy or deterioration in its active components.

The cannula in accordance with the invention is particularly practical to use, because the two products are ejected directly into the vagina almost simultaneously with a simple manoeuvre.

Various types of cannula already exist which enable a drug, a douching fluid or the like to be introduced into the vagina.

For example, devices exist which consist of a small plastic reservoir connected to a cannula containing a set of holes in the end through which the product is expelled.

When the cannula is fully screwed onto the body of the container, the end of the cannula perforates a closing wall, thus allowing expulsion of the product, performed by manually compressing the container, which acts as a pump.

However, all the known devices only allow the administration of a single product, which may be in liquid, cream or another form.

The present invention falls into this sector, being designed to solve the problem of administering two products simultaneously, and keeping the said products completely separate until the time of use. The said products may both be semi-solid products; alternatively, as in the case which will be illustrated herein in detail, one product may be in gel form and the other in tablet form.

For this purpose, the invention comprises a cannula fitted at the anterior extremity with a seating designed to house a tablet, a separator able to slide inside the cannula and fitted with means designed to engage the said tablet, a set of holes in a section of the cannula wall upstream of the separator, to allow expulsion of the gel contained in the cannula, and a plunger which expels the gel during the first part of its travel and subsequently engages the separator, causing it to engage with and expel the tablet.

This invention will now be described in detail, by way of example but not of limitation, by reference to the annexed figures in which:

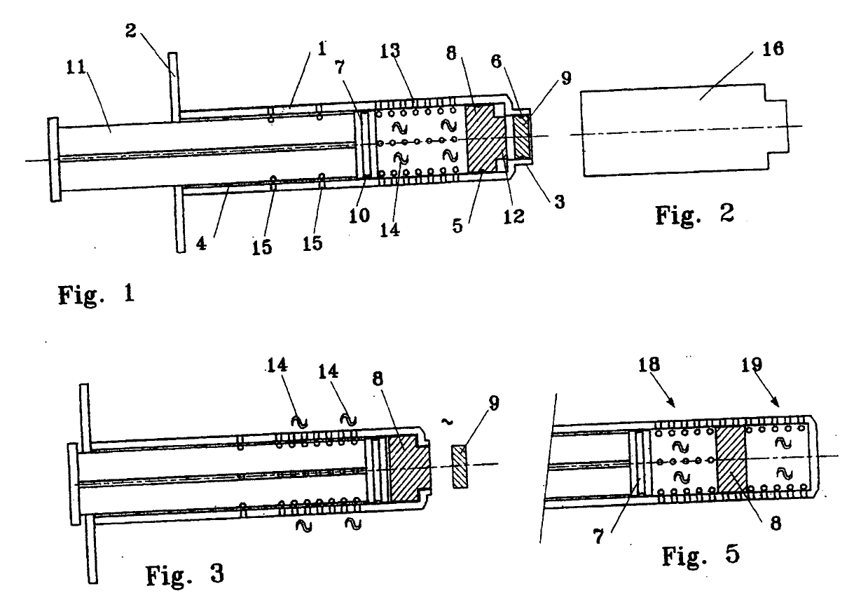

FIG. 1 shows a cross-section of a cannula in accordance with the invention before use, with the packaged product

FIG. 2 shows a cannula sheath which acts as the packaging of the product

FIG. 3 shows a cross-section of a cannula in accordance with the invention after expulsion of the products

FIG. 4 is a perspective view of the cannula in accordance with the invention

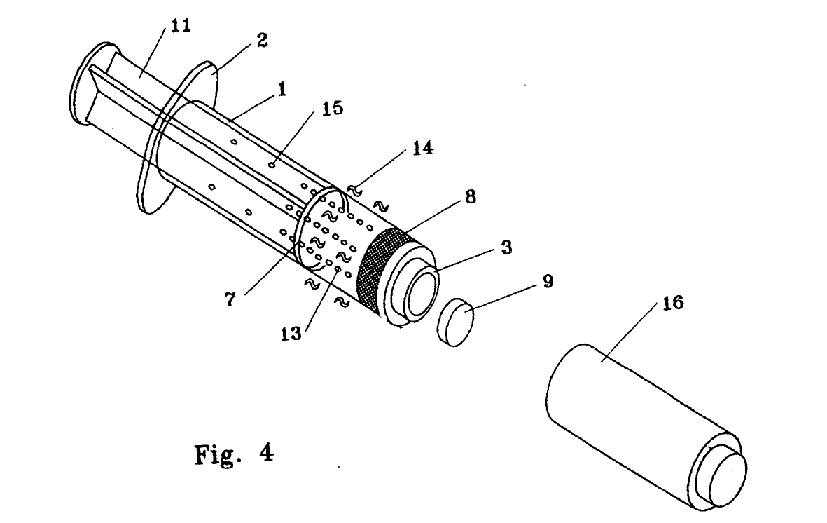

FIG. 5 shows a cross-section of a further preferred embodiment of the cannula in accordance with the invention.

As shown in the annexed figures, the cannula in accordance with the invention comprises a barrel 1 which is open at both ends and fitted on one side (the proximal end) with a pair of wings or the like 2, and on the opposite side (the distal end) with a collar of smaller diameter, shown as 3, which surrounds the aperture of the cannula.

On the inner wall of barrel 1 there are three facing ridges shown as nos. 4, 5 and 6 which are designed to engage a plunger 7, a separator/ejector element 8, and a tablet 9, destined to be introduced into the vagina, respectively.

Plunger 7, which slides tightly inside barrel 1, could contain a ring-shaped groove 10 which engages with ridges 4 (or a corresponding raised ring) to retain the plunger and the plunger rod 11 in position before use of the cannula.

Separator 8 consists of a substantially cylindrical body which forms a seal against the inner wall of the cannula and in its anterior part has a cylindrical section 12 of smaller diameter, especially a diameter substantially corresponding to the inner diameter of collar 3.

The length of section 12 is substantially equal to the length of collar 3, so that when separator 8 is in contact with the distal closing wall of the cannula (position illustrated in FIG. 3), cylindrical body 12 does not project from the cannula.

The body of separator 8 too, will preferably have a ring-shaped groove designed to engage with ridges 5.

In accordance with the invention, the wall of barrel 1 presents a plurality of holes 13 distributed along a length of wall immediately upstream of separator 8, in correspondence with the distal end. These holes allow the expulsion of a semi-solid product, for example a drug or a pharmaceutically acceptable carrier in the form of a gel 14, contained in the cannula.

A second section of cannula wall, upstream of the preceding one, presents a set of holes 15 through which air is expelled from the cannula during the stage of filling the cannula with the drug in gel form, so that plunger 7 can slide until it comes into contact with the gel without any air being trapped.

The cannula is completed by a sheath 16 which acts as the packaging for the product and at the time prevents the gel from leaking out of expulsion holes 13 of the cannula during the filling stage.

The dimensions of the cannula can vary, depending on the material to be delivered, although for most applications its length could be approx. 12 centimetres and its diameter approx. 12-14 mm.

In order to prepare the cannula containing the product, the separator is first inserted into the barrel until it engages with the corresponding ridge 5, which retains it at a distance from the distal end of the cannula, in the position shown in FIG. 1.

Next, the tablet is introduced by inserting it into collar 3, where it is retained by ridges 6.

Sheath 16 is then fitted over the cannula so as to close the end containing the tablet and cover the entire length containing holes 13.

Next, the required amount of gel is introduced into the cannula, in contact with separator 8, and plunger 7 is inserted and pushed in until it comes into contact with the gel.

The plunger, which is made of plastic suitable for the purpose, slides tightly along the inner wall of the barrel, as in the case of a syringe.

In order to use the cannula it is sufficient to remove sheath 16, hold the cannula by wings 2, and press on rod 11 of plunger 7, which is forced to advance into the cannula.

The thrust exerted by plunger 7 first forces out gel 14 through holes 13 in the cannula wall.

When the plunger has pushed all of the gel out of the cannula, it will come into contact with separator/ejector system 8.

The subsequent advance of the plunger will force the separator out of its seating, pushing it forward until cylindrical body 12 penetrates into collar 3, removing and expelling vaginal tablet 9, which is thus deposited in the vagina.

At this point, separator 8 engages with anterior wall of the cannula, preventing it from advancing further.

The dimensions of ridges 4, 5 and 6, and especially those of ridge 5, which retains separator 8 in position, will be chosen according to the characteristics of the gel, in such a way that the force required to move the separator is greater than that needed to expel the gel.

As will clearly appear from the description supplied, the cannula in accordance with the invention is particularly convenient and practical, since it allows the delivery of two products with different physical characteristics, such as a product in gel form and a solid product in tablet form.

In accordance with a different form of embodiment of the same idea, illustrated in FIG. 5, the anterior extremity of the cannula could be closed, and separator 8 could act as a partition wall between two consecutive lengths of the cannula shown as 18 and 19, which could be filled with two different gels.

The cannula could also be used to deliver different products for different clinical applications, including, by way of example but not of limitation, the following:

1—Acidifying gel and vaginal tablet based on Lactobacillus acidophilus to rebalance the vaginal ecosystem.

2—Acidifying gel and metronidazole tablet to treat vaginitis caused by Gardnerella vaginalis and Trichomonas and simultaneously restore the normal vaginal ecosystem.

3—Gel with a slightly acid pH and antifungal vaginal tablets to treat fungal vaginitis and provide a soothing effect on the damaged mucosa.

4—Acidifying gel and antibiotic tablets such as meclocycline to treat bacterial vaginitis and rebalance the vaginal ecosystem.

5—Oestradiol hormone gel and Lactobacillus acidophilus vaginal tablet for topical treatment of the menopause and dystrophic forms, and restoration of the normal vaginal ecosystem.

1. Disposable vaginal cannula designed to deliver drugs with different compositions simultaneously, characterised in that it comprises means (1, 3) designed to receive two or more different products to be administered and means (7) designed to expel the said products simultaneously or successively.

2. Disposable vaginal cannula designed to deliver drugs with different compositions simultaneously, as claimed in claim 1, characterised in that it comprises a barrel (1) designed to contain a predetermined quantity of two or more products, means (7) designed to exert a thrust on the said products to expel them, a set of openings (3, 13) being provided in correspondence with the parts of the said container in which the said products are situated, which allow the products to exit as a result of the thrust exerted on them.

3. Disposable vaginal cannula as claimed in claim 2, characterised in that the said barrel (1) is fitted with a separator (8) designed to prevent the said products from coming into contact with one another before use, the said separator (8) being able to move inside the said container (I) so that it slides along the cannula as a result of the thrust applied to the products.

4. Disposable vaginal cannula as claimed in claim 4, characterised in that it has one open end with a seating (3) designed to contain a tablet (9) of a product to be administered, said separator (8) which can slide inside the said cannula, and being equipped with means (12) designed to engage the said tablet to control its expulsion.

5. Disposable vaginal cannula as claimed in claim 1, characterised in that it comprises:
a barrel (1) designed to contain a dose of a semi-solid product (14)
a plunger (7) designed to press on the said product to control its expulsion
a plurality of holes (13) in the wall of the said barrel (1), in correspondence with the distal section of the cannula, to allow the exit of the said semi-solid product (14);
a sealed separator (8), inserted into the cannula downstream of the said semi-solid product (14), which said separator (8) has a projecting anterior part (12)
an opening in the anterior wall of the cannula provided with means (3) designed to receive and retain a tablet (9) of a second medical product, the anterior section of which said separator (8) being fitted with projecting parts (12) designed to engage with and eject the said tablet as a result of the movement of the said separator.

6. Cannula as claimed in each of the preceding claims, characterised in that it is provided with a covering sheath (16) designed to be fitted over the distal end of the cannula, which said sheath covers at least the length of cannula containing the said openings (3, 13) for the exit of the product.

7. Vaginal cannula as claimed in each of the preceding claims, characterised in that the inner wall of the said cannula presents ridges (5) designed to retain the said separator in position.

8. Cannula as claimed in claim 5, characterised in that the inner surface of the cannula presents ridges (6) designed to retain the tablet in position.

9. Cannula as claimed in claim 5, characterised in that the inner surface of the cannula presents means (4) designed to retain plunger (7), which controls the expulsion of the product, in position prior to use.

10. Cannula as claimed in each of the preceding claims, characterised in that the wall of the cannula upstream of the section containing the semi-solid product contains a series of holes (15) designed to allow the exit of air when plunger (7) is introduced into the cannula.

11. Disposable vaginal cannula designed to deliver drugs with different compositions simultaneously, as claimed in claim 1, characterised in that at least one section of the wall of the said barrel (1), in correspondence with a closed extremity thereof, contains holes (18, 19) for the exit of two different products, the said separator (8) being inserted between the said two sets of holes.
